(19) United States
(12) Patent Application Publication
(10) Pub. No.: US 2024/0194314 A1
(43) Pub. Date: Jun. 13, 2024

(54) MEDICAL CARE ASSISTANCE SYSTEM, MEDICAL CARE ASSISTANCE DEVICE, AND PROGRAM

(71) Applicant: OMRON HEALTHCARE Co., Ltd., Kyoto (JP)

(72) Inventors: Hironori SATO, Kyoto (JP); Yoko KANEMITSU, Kyoto (JP); Fumihiko NAKAMURA, Kyoto (JP); Waka UEDA, Kyoto (JP); Takahiro HAMAGUCHI, Kyoto (JP); Rika KATO, Kyoto (JP)

(21) Appl. No.: 18/442,711

(22) Filed: Feb. 15, 2024

Related U.S. Application Data
(63) Continuation of application No. PCT/JP2022/044959, filed on Dec. 6, 2022.

(30) Foreign Application Priority Data
Dec. 15, 2021 (JP) 2021-203296

(51) Int. Cl. G16H 15/00; A61B 5/021; A61B 5/024; G16H 10/60; G16H 20/10

(52) U.S. Cl. CPC G16H 15/00; A61B 5/021; A61B 5/024; G16H 10/60; G16H 20/10

(57) ABSTRACT

A medical care assistance system includes measured value acquisition means configured to acquire a measured value related to biological information of a patient, medication information acquisition means configured to acquire medication information that is related to medicine prescribed to the patient, and includes information of a first prescription start date that is an earliest date of prescription of medicine with at least one prescription content, average measured value calculation means configured to calculate an average measured value that is an average value of the measured value of the patient in each predetermined period, medicinal effect confirmation support image generation means configured to generate a medicinal effect confirmation support image including measured value transition information indicating transition of the average measured value in time series, in each of the predetermined periods before and after the first predetermined period, and output means configured to output the medicinal effect confirmation support image.

### CROSS-REFERENCE TO RELATED APPLICATIONS

This application is the U.S. national stage application filed pursuant to 35 U.S.C. 365(c) and 120 as a continuation of International Patent Application No. PCT/JP2022/044959, filed Dec. 6, 2022, which application claims priority to Japanese Patent Application No. 2021-203296, filed Dec. 15, 2021, which applications are incorporated herein by reference in their entireties.

### TECHNICAL FIELD

The present invention belongs to a technical field related to healthcare, and relates to a medical care assistance system, a medical care assistance device, and a program.

### BACKGROUND ART

In recent years, there has been an increasing need for remote medical care, and as a part of this, there has been proposed a medical care assistance system in which biological information, such as a blood glucose level or a blood pressure value measured by a patient at home, is transmitted to a database, and a doctor considers changing a medication while checking the measurement results (for example, Patent Documents 1 and 2).

Patent Documents 1 and 2 disclose a medication support device that acquires each of medication information including a type and an amount of medicine, blood pressure measurement information of a patient, and dosing information indicating a dosing status of the patient, comprehensively associates these pieces of information in time series, and displays medication treatment results.

According to such a device, the doctor can adopt an appropriate treatment strategy by determining the effect of the medicine by comprehensively associating the information on the medicine prescribed to the patient with the patient's dosing status and the blood pressure measured value.

### CITATION LIST

### Patent Literature

Patent Document 1: JP 2018-151993 A
Patent Document 2: JP 2019-207536 A

### SUMMARY OF INVENTION

### Technical Problem

However, the known apparatus (system) as described in the above-mentioned patent literature has a problem in that after a change in prescription, the effect of such a change is obscured by daily (that is, short-term) blood pressure fluctuation meaning that it is difficult to figure out whether the prescription change has started to become effective, and the level of such an effect. In addition, in the case of so-called masked hypertension or white coat hypertension, which are occurrence of a blood pressure difference due to a difference in measurement location, it is further difficult to determine the medicinal effect.

In view of the above-described problems, the present invention relates to a system related to medical care assistance, and an object thereof is to provide a technique enabling a medical professional to reliably and easily confirm the effect of medication.

### Solution to Problem

The present invention adopts the following configurations to solve the above-described problems. Specifically, a medical care assistance system includes:
measured value acquisition means configured to acquire a measured value related to biological information of a patient;
medication information acquisition means configured to acquire medication information that is related to medicine prescribed to the patient, and includes information of a first prescription start date that is an earliest date of prescription of medicine with at least one prescription content;
average measured value calculation means configured to calculate an average measured value that is an average value of the measured value of the patient in each predetermined period;
medicinal effect confirmation support image generation means configured to generate a medicinal effect confirmation support image including measured value transition information indicating transition of the average measured value in time series, in each of the predetermined periods before and after the first predetermined period; and
output means configured to output the medicinal effect confirmation support image.

Here, the “predetermined period” is not particularly limited as long as it is a period effective for confirmation of medicinal effect, and can be, for example, one week or more. Also, the “output means” may be a display device, such as a liquid crystal display, or may be a printing device, such as a printer.

With such a configuration, prescription start (or change. The same applies hereinafter), and the average value of the biological information for each predetermined period thereafter can be compared with the average value of the biological information for each predetermined period before the reference date. That is, since the medical professional can compare the average values with the daily variations of the measured values of the biological information within the predetermined period from the start of the prescription smoothed, by checking the medicinal effect confirmation support image, and thus can easily and reliably confirm the effect of the medication.

The measured value acquisition means may further acquire information related to a time period and/or a location at which the biological information is measured, the average measured value calculation means may calculate the average measured value for each time period and/or location at which the biological information is measured, and the measured value transition information in the medicinal effect confirmation support image may indicate a change in the average measured value for each time period and/or location at which the biological information is measured.

Here, the “time period” can be a period such as, for example, morning, daytime, evening, and night. In the case of measuring blood pressure, for example, it is recommended to perform the measurement twice, after getting up (before breakfast) and before going to bed. Therefore, preferably, measurement data can be managed for each of the time periods as described above. Note that “information related to the time period” may be, for example, time data and the like indicating a date and time, and the time period may be designated by a range including the time. The “location” mentioned herein can be a classification of a range that may affect the measured value of the biological information, such as a home (family) and a medical examination room. Note that “information related to the location” may be, for example, GPS positioning information or the like, and the “location” may be designated by the positioning information.

In this way, by calculating the average measured value for a difference in each of time period and location, it is possible to display the transition of the biological information before and after the start of the prescription, with distinction between the increase and decrease components of the measured value caused by the difference in the time period and the location of the biological information. Therefore, the medical professional can more appropriately determine the medicinal effect.

The medical care assistance system may further include target achievement determination means configured to determine whether an improvement target of the biological information of the patient set in advance for each of the average measured values is achieved, and the measured value transition information in the medicinal effect confirmation support image may indicate information on whether the improvement target is achieved for each of the average measured values.

Here, an “improvement target value” may be a general-purpose so-called normal value or the like, or may be a value decided each time according to the individual patient's state, a prescription, or the like. Further, it may be an absolute value of the measured value itself of the biological information, or a relative value which is an increase/decrease target value based on the measured value at a certain point in time. The “information on whether the target value is achieved” can be, for example, a text message, a color, a symbol (mark), a line segment on a graph indicating the target value, or the like. With such a configuration, the medical professional can easily confirm whether the improvement target value is achieved, and can more quickly determine the medicinal effect.

The medical care assistance system may further include change amount calculation means configured to calculate an increasing/decreasing change amount of the average measured value in one of the predetermined periods from the average measured value in an immediately preceding one of the predetermined periods, and the measured value transition information in the medicinal effect confirmation support image visibly may indicate the increasing/decreasing change amount for each of the average measured values, with increase and decrease being distinguishable from each other.

Here, whether the change amount is increased or decreased can be indicated by, for example, a text message, a color, a symbol (mark), or the like. With such a configuration, the medical professional can easily check the amount of change in the average measured value, and can more quickly determine the medicinal effect.

The medical care assistance system may further include reliability determination means configured to determine whether the average measured value has a predetermined reliability, and the measured value transition information in the medicinal effect confirmation support image may indicate, in a distinguishable manner, the reliability of each of the average measured values. The reliability determination means may make the determination on the reliability based on whether the number of times the biological information is measured in the predetermined period satisfies a predetermined criterion.

Here, the reliability can be indicated by a text message, a color, a symbol (mark), or the like. Whether the number of times of measurement satisfies the predetermined criterion may be determined for each time period or each location. With such a configuration, when the reliability of the average measured value is low (due to a small number of times of measurement or the like), the medical professional can recognize the low reliability and thus can make a determination on the assumption that the reliability is low.

The measured value acquisition means may acquire, when an abnormality occurs while the biological information is being measured, information on the abnormality together with the measured value, the average measured value calculation means may calculate an average value of measured values in the predetermined period, while excluding a measured value corresponding to the occurrence of the abnormality, and the reliability determination means may determine whether the predetermined criterion is satisfied, by subtracting from the number of times of the measurement, measurement of the biological information corresponding to the occurrence of the abnormality.

Here, the abnormality while the biological information is being measured can include, for example, a body motion, an arrhythmia, or the like that affects the measurement. With such a configuration, it is possible to prevent the reliability of the average measured value from being adversely affected by inclusion of inappropriate measurement data.

The medication information may include information related to a name of the prescribed medicine and an amount of the prescribed medicine, and the medicinal effect confirmation support image may indicate medicine-related information including the medication information in addition to the measured value transition information.

With such a configuration, the medical professional can confirm the relationship between the prescribed medicine and the medicinal effect thereof in a list, and can quickly review the treatment strategy.

The medication information may include, when there is a medicine prescribed to the patient with another content in addition to the medicine prescribed with the one prescription content, information related to a name and an amount of the medicine with the other content, and
when a period indicated by the measured value transition information displayed in the medicinal effect confirmation support image includes a period during which the medicine is prescribed with the other content, the medicine-related information may further indicate information related to the name and the amount of the medicine in the period during which the medicine is prescribed with the other content.

With such a configuration, it is possible to check the medication information before and after the prescription change in a list, and it is possible to reduce the burden on the medical professional.

The medication information may include information of a second prescription start data that is earliest date of the prescription of the medicine with the other content, and
when a period displayed in the medicinal effect confirmation support image includes the second prescription start date, a mark indicating the second prescription start date may be displayed in the medicinal effect confirmation support image.

The second prescription start date may be, in time series, a date before the first prescription start date or a date after the first prescription start date. With such a configuration, it is possible to suggest to the medical professional that it is possible to check the measured value transition information with the second prescription start date used as a reference.

The medical care assistance system may further include dosing related information acquisition means configured to acquire dosing information related to a dosing status of the patient and/or side effect information related to a side effect as a result of dosing by the patient, and the medicine-related information in the medicinal effect confirmation support image may indicate the dosing information and/or the side effect information.

Here, the dosing information can indicate, for example, whether the medication is taken, the dosing adherence rate, or the like, and the side effect information can include the type, frequency, or the like of the side effect. For example, as a function of an application of a portable information processing terminal, such as a smartphone used by a patient, it is possible to acquire, as dosing information, and side effect information, subjective information from the patient himself/herself through a question and answer with a so-called chatbot. However, the information acquisition means is not limited to such a mode, and may be any means. As another example, a sensor that detects removal of a medicine may be provided in a container in which the patient stores the medicine, and the dosing information may be acquired from information (the amount of the medicine removed, and the date and time of the removal) acquired by the sensor. Also, the patient may be linked with a database of a pharmacy from which the medicine is purchased, and the dosing information may be acquired from the sales history.

With such a configuration, the dosing information and the side effect information can be confirmed by the medical professional with excellent perspicuity, and can be useful for examining an appropriate treatment strategy.

In addition, the measured value transition information can include a graph display indicating a transition of the average measured value of the biological information. Such a configuration enables more intuitive recognition of the transition of the average measured value, to contribute to the medical professional's medicinal effect determination.

The measured value of the biological information may include a blood pressure value and a pulse rate, and the graph display may display average values of the blood pressure value and the pulse rate measured in the one predetermined period on the same axis.

The present invention can also be recognized as a medical care assistance device, including: the measured value acquisition means; the medication information acquisition means; the average measured value calculation means; and the medicinal effect confirmation support image generation means, the medical care assistance device constituting at least a part of the medical care assistance system.

Further, the present invention can also be considered as a program for causing a computer to function as such a medical care assistance device, and a computer-readable recording medium including such a program recorded therein in a non-transitory manner.

Also, the configurations and processing described above can be combined with one another to constitute the present invention unless the combination leads to contradiction.

### Advantageous Effects of Invention

The present invention relates to a system related to medical care assistance, and can provide a technique enabling a medical professional to reliably and easily confirm the effect of medication.

### BRIEF DESCRIPTION OF DRAWINGS

FIG. 1 is a schematic diagram illustrating a configuration of a medical care assistance system according to an embodiment.

FIG. 2 is a flowchart schematically illustrating a flow of remote medical care performed in the embodiment.

FIG. 3 is a block diagram illustrating a functional configuration of a server device according to the embodiment.

FIG. 4 is a block diagram illustrating a functional configuration of a doctor-side terminal according to the embodiment.

FIG. 5 is a first diagram illustrating a screen example output on the doctor-side terminal.

FIG. 6 is a second diagram illustrating a screen example output on the doctor-side terminal.

FIG. 7 is a third diagram illustrating a screen example output on the doctor-side terminal.

FIG. 8 is a block diagram illustrating a functional configuration of a patient-side terminal according to the embodiment.

FIG. 9(A) is a first diagram illustrating a screen example at the time of executing an automatic medical interview program according to the embodiment. FIG. 9(B) is a second diagram illustrating a screen example at the time of executing the automatic medical interview program according to the embodiment.

FIG. 10 is a diagram illustrating a flow of information exchange and processing performed in the medical care assistance system according to the embodiment.

FIG. 11 is a first diagram illustrating a screen example output on a doctor-side terminal according to a modified example.

FIG. 12 is a second diagram illustrating a screen example output on the doctor-side terminal according to the modified example.

FIG. 13 is a diagram illustrating an overview of a medical care assistance system according to another embodiment.

### DESCRIPTION OF EMBODIMENTS

### Example 1

Embodiments of the present invention will be specifically described below with reference to the drawings. However, it should be noted that the dimension, shape, relative arrangement and the like of the components described in this embodiment are not intended to limit the scope of this invention to them alone, unless otherwise stated.

### System Configuration

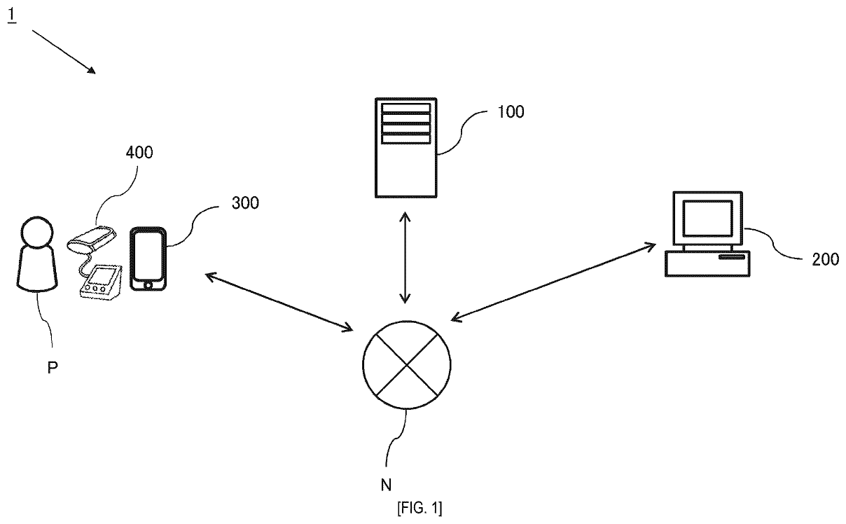

FIG. 1 is a schematic diagram illustrating a configuration of a medical care assistance system 1 according to the present example. As illustrated in FIG. 1, the medical care assistance system 1 includes a server device 100, a doctor-side terminal 200 used by a doctor, and a patient-side terminal 300 and a blood pressure monitor 400 used by a patient P. These respective configurations can communicate with one another via a communication network N.

The medical care assistance system 1 according to the present embodiment is a system used for remote medical care. The medical care assistance system 1 transmits a measured value of biological information such as a blood pressure value measured by a patient at home to the server device 100 via the communication network N, processes the information, and provides the processed information to a medical professional, thereby assisting the doctor in performing treatment on the patient.

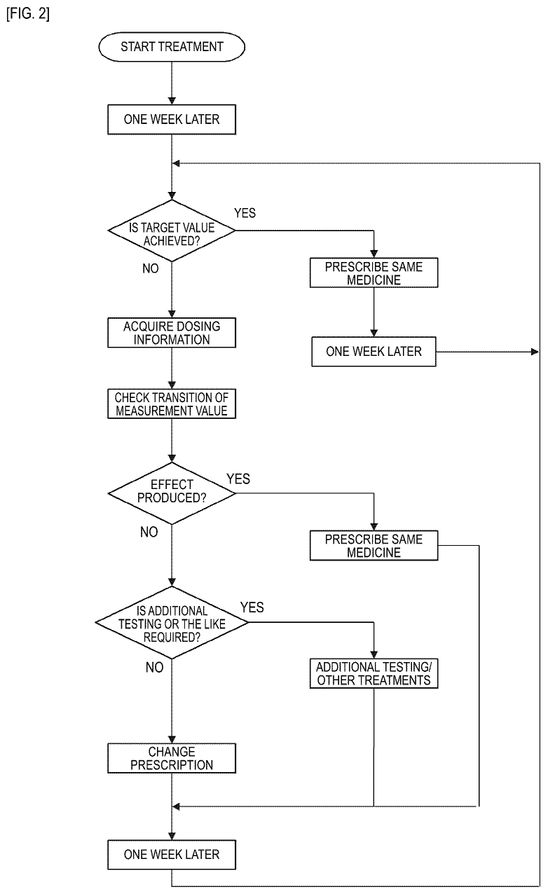

FIG. 2 is a flowchart schematically illustrating a flow of the remote medical care performed in the present embodiment. Hereinafter, the remote medical care performed in the present embodiment will be specifically described with reference to FIG. 2. That is, a patient who has received a definite diagnosis of hypertension starts medication treatment in accordance with a doctor's prescription, and continuously measures his/her blood pressure at home. The medical care assistance system 1 collects the measured values, and after a predetermined period has elapsed (here, one week), determines whether the average value of the measured values within the predetermined period (hereinafter, referred to as an average measured value) has achieved a target value set in advance (hereinafter, the fact that the average measured value has achieved the target value is also expressed as “being under control” or the like). Here, the average measured value achieving the target value means, for example, that the (average) blood pressure value is less than a predetermined threshold. In a state where the average measured value is under control, the same medicine continues to be taken, and after a predetermined period of time (here, one week) has elapsed, it is determined whether the average measured value of blood pressure is in a controlled state.

On the other hand, when the target value is not achieved, the medication state is checked to see whether the patient has been taking the medicine according to the prescription. In addition, the transition of the average measured value at each predetermined period is checked by the doctor, and is used for deciding on a treatment strategy together with the information on the medication state. The confirmation of the medication information may be periodically performed regardless of whether the control of the average measured value has succeeded.

When the doctor checks the state of transition of the average measured value and determines that a certain effect is produced by the medication, the doctor decides to continue to administer the same medicine (wait and see). In that case, it is determined again after a predetermined period (here, one week) has elapsed, whether the average measured value of blood pressure is in a controlled state.

Also, upon determining that the medication does not seem to be effective, the doctor considers the possibility of other diseases and decides to perform additional testing or treatment (including change in prescription) taking into account the other diseases. In that case, it is determined again after a predetermined period (here, one week) has elapsed, whether the average measured value of blood pressure is in a controlled state.

In addition, when other diseases are not the causes and the state is expected to improve by changing the medicine being taken, such as stopping the medication due to a side effect, the prescription contents are changed. In that case, it is determined again after a predetermined period (here, one week) has elapsed, whether the average measured value of blood pressure is in a controlled state.

By repeatedly performing the flow described above, the remote medical care of the hypertensive patient is executed.

The medical care assistance system 1 according to the present embodiment supports the execution of such a flow, and reduces the burden on the doctor (medical professional). Hereinafter, the respective configurations of the system will be described in detail.

### Server Device

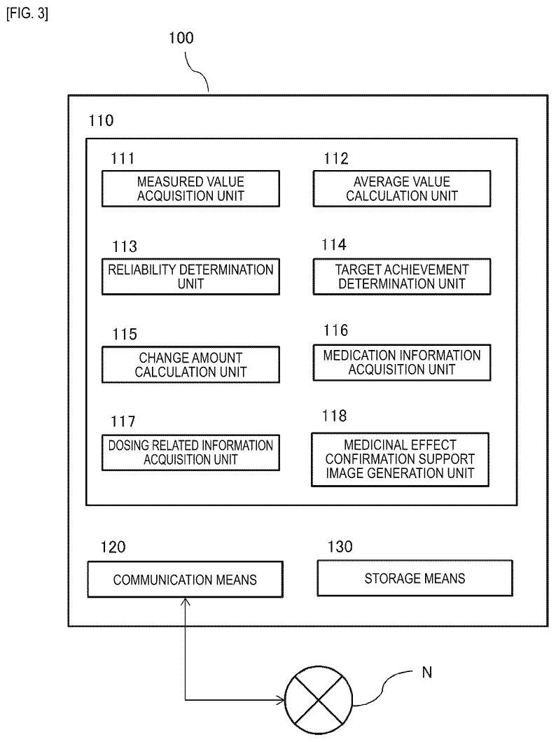

FIG. 3 is a block diagram illustrating a functional configuration of the server device 100. The server device 100 is constituted of a general server computer and, as illustrated in FIG. 3, includes a control unit 110, communication means 120, and storage means 130.

The control unit 110 is means that manages the control of the server device 100, and is constituted of a processor, such as a Central Processing Unit (CPU) or a Digital Signal Processor (DSP). The control unit 110 includes, as functional modules related to biological information management, functional units including a measured value acquisition unit 111, an average value calculation unit 112, a reliability determination unit 113, a target achievement determination unit 114, a change amount calculation unit 115, a medication information acquisition unit 116, a dosing related information acquisition unit 117, and a medicinal effect confirmation support image generation unit 118. These respective functional units will be described in detail below.

The communication means 120 is communication means for connecting the server device 100 to the communication network N, and is constituted including, for example, a communication interface board and a wireless communication circuit for wireless communication.

Although not illustrated, the storage means 130 includes a main storage unit, such as a Read only memory (ROM) or a Random access memory (RAM), and an auxiliary storage unit, such as an EPROM, a Hard Disk Drive (HDD), a Solid State Device (SSD), or a removable medium. The auxiliary storage unit stores an operating system (OS), various programs, and the like. By the stored programs being loaded into a work area of the main storage unit and executed, and the respective component units and the like being controlled by the execution of the programs, the respective functional units that meet predetermined purposes can be realized.

The measured value acquisition unit 111 acquires, via the communication network N, a measured value (such as, for example, systolic blood pressure value, diastolic blood pressure value, and pulse rate) of the biological information measured by the patient P using the blood pressure monitor 400, and stores the measured value in the storage means 130. The information acquired by the measured value acquisition unit 111 includes not only the measured value of the biological information but also information related to a time period in which the measurement is performed (for example, time data), information related to a location where the measurement is performed (for example, a difference between a home and a medical examination room, or the like), and information related to an abnormality at the time of measurement that adversely affects the reliability of the measured value (for example, body motion exceeding an allowable range, presence of an irregular pulse wave, or the like).

The average value calculation unit 112 calculates an average measured value which is an average value of the acquired measured values within a predetermined period. When there are a plurality of time periods and locations at which the biological information is measured, an average measured value is calculated for each of the time periods and locations. In addition, in a case where the abnormality at the time of measurement as described above is included, the average value may be calculated by excluding the measured value corresponding to the time of occurrence of the abnormality. The calculated average value is stored in the storage means 130.

The reliability determination unit 113 determines whether the calculated average measured value has certain reliability. Specifically, the reliability may be determined based on whether the number of blood pressure measurements (and/or measurement opportunities) within a predetermined period has reached a certain number of times, for example. In addition, in a case where the abnormality at the time of measurement as described above is included, the number of times of measurement can be counted without counting the measurement at the time of occurrence of the abnormality.

The target achievement determination unit 114 determines, for each calculated average measured value, whether an improvement target set in advance for the individual patient P is achieved. Specifically, the determination may be made based on whether the average measured value deviates from a predetermined upper limit (lower limit) threshold serving as the improvement target value. The result of the determination indicates whether the blood pressure value of the patient P is under control. The improvement target value may be stored in advance in the storage means 130, for example.

The change amount calculation unit 115 compares an average measured value in a certain predetermined period (for example, one week) with an average measured value in a predetermined period immediately preceding the certain predetermined period, and calculates an increasing/decreasing change amount from the average measured value in the immediately preceding certain predetermined period. The calculated value is stored in the storage means 130.

The medication information acquisition unit 116 acquires medication information related to a name of a medicine prescribed to the patient P, an amount of each medicine, a start date of the prescription with the prescription content, and the like. The medication information may be acquired by, for example, reading out the information from the storage means 130 storing the medication information including the past history. In addition, the medication information may be acquired from the doctor-side terminal 200 to be described later, an electronic medical chart management system (not illustrated), or the like, via communication means 120.

The dosing related information acquisition unit 117 acquires dosing related information including medication-related information including dosing information related to whether medication is provided to the patient and the dosing adherence rate (dosing frequency), and side effect information related to the content and frequency of side effects at the time of medication and the like, via the patient-side terminal 300 as described later, for example. The timing at which the dosing related information is acquired can be, for example, a timing such as that when the target achievement determination unit 114 determines that the improvement target is not achieved. However, this should not be construed in a limiting sense, and the dosing related information may be periodically acquired.

The medicinal effect confirmation support image generation unit 118 generates a medicinal effect confirmation support image in which measured value transition information indicating a change in the average measured value calculated by the average value calculation unit 112 for each predetermined period, as well as medication information and dosing related information (the medication information and the dosing related information will be hereinafter collectively referred to as medicine-related information) are browsable. Specifically, the display content related to the measured value transition information and the medicine-related information is specified based on the data output from each of the functional units including the measured value acquisition unit 111, the average value calculation unit 112, the reliability determination unit 113, the target achievement determination unit 114, the change amount calculation unit 115, the medication information acquisition unit 116, and the dosing related information acquisition unit 117, and stored in the storage means 130. For example, in the measured value transition information, for each average measured value to be displayed, information related to the reliability of the average measured value, whether the improvement target is achieved, the amount of change from the immediately preceding average measured value, and the like is indicated. The generated medicinal effect confirmation support image is transmitted to the doctor-side terminal 200 via the communication network N. Details of the medicinal effect confirmation support image will be described later.

### Doctor-Side Terminal

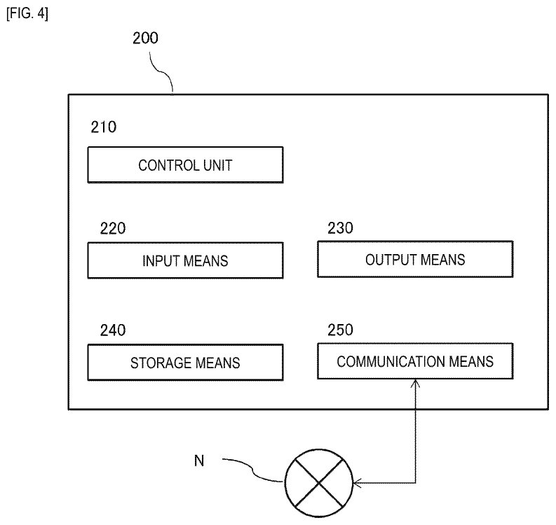

FIG. 4 is a block diagram illustrating a functional configuration the doctor-side terminal 200. The doctor-side terminal 200 is a general computer, such as a fixed personal computer, a portable notebook-type personal computer, or a tablet device, and includes a control unit 210, input means 220, output means 230, storage means 240, and communication means 250.

The control unit 210 is means for controlling the doctor-side terminal 200, and is constituted of, for example, a CPU. The input means 220 is means that receives information input from the outside, such as a keyboard, a mouse, a touch panel, a camera, or a microphone. The output means 230 is constituted including a liquid crystal display, a speaker, and the like. The storage means 240 is constituted similarly to the server device including a main storage unit, an auxiliary storage unit, and the like, and stores various data acquired via the communication network N, such as an operating system (OS) and various programs. The communication means 250 is constituted including, for example, a communication interface board and a wireless communication circuit for wireless communication.

Note that, although not illustrated, the doctor-side terminal may be accessible to an electronic medical chart management system. In such a case, electronic medical chart data of a patient stored in the electronic medical chart management system may be read out to be transmitted to the server device 100, and information transmitted from the server device 100 may be associated with the electronic medical chart data. In such a case, the doctor can also check the medicinal effect confirmation support image via the electronic medical chart management system.

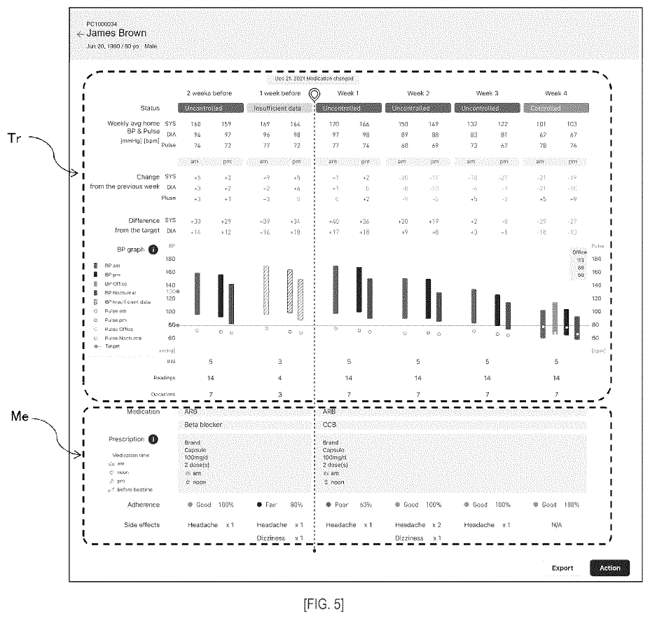

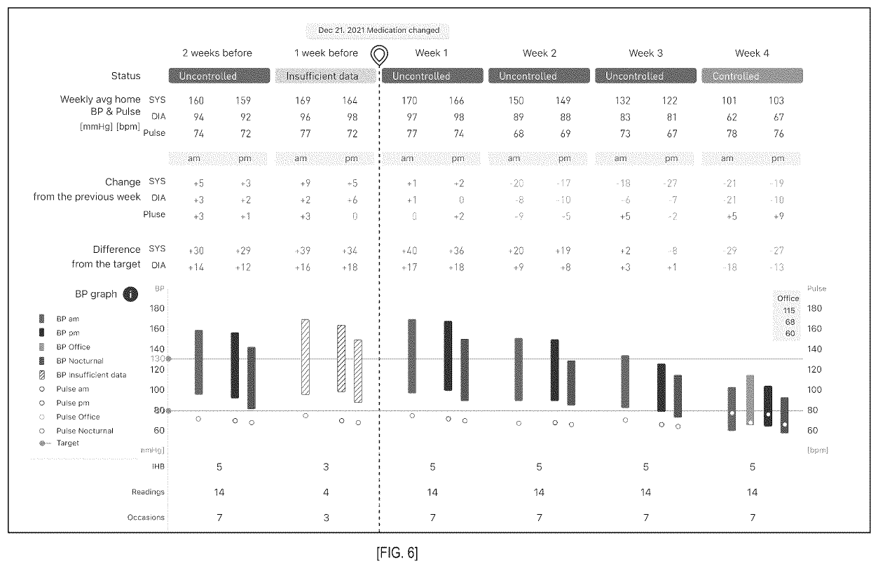

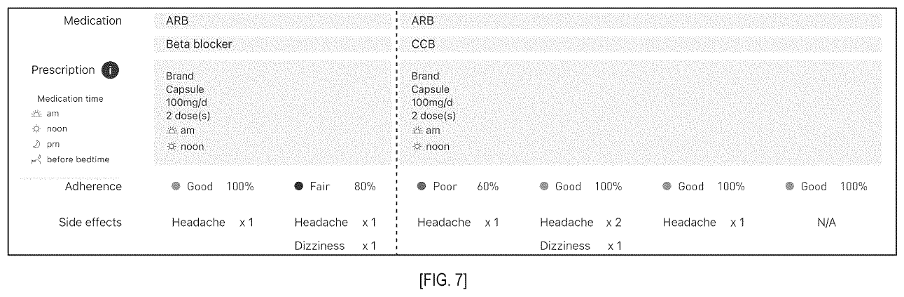

In the doctor-side terminal 200, the medicinal effect confirmation support image are acquired from the server device 100 via the communication network N, and these pieces of information are output to the output means 230. FIG. 5 to FIG. 7 illustrate examples of screens displayed on the output means 230 of the doctor-side terminal 200. FIG. 5 is an explanatory diagram illustrating an example of a medicinal effect confirmation support image for one patient P taken care of by the doctor who manages the terminal. As illustrated in FIG. 5, the medicinal effect confirmation support image is an image in which information related to patient attributes such as patient name, gender, and age is displayed in the top row, measured value transition information Tr is displayed in the middle row, and medicine-related information Me is displayed in the bottom row. FIG. 6 is an enlarged view illustrating part of the measured value transition information Tr, and FIG. 7 is an enlarged view illustrating part of the medicine-related information Me.

As illustrated in FIG. 5 and FIG. 7, in the measured value transition information Tr in the present embodiment, the average values (weekly avg home BP & Pulse) of the systolic blood pressure (SYS), the diastolic blood pressure (DIA), and pulse rate (Pulse) in each of a week before and a week after a date of change (Dec. 21, 2021) in prescription content are displayed in time series. More specifically, respective average values of home SYS and DIA blood pressures (blood pressure values measured at home) in a week are displayed for each of the time periods in am (after getting up) and pm (before going to bed). In the present embodiment, the predetermined period is divided with the prescription change date is used as a reference, and the day after the change date is used as the initial calculation date (meaning that the prescription change date is included in the preceding week period). This way of dividing is preferable because it is expected that the dosing content does not change at the day when the prescription is changed, but is actually changed the next day in many cases.

In addition, in the measured value transition information Tr, a change amount (Change from the previous week) in a case of comparing with a numerical value of the previous week and a value (Difference from the target) indicating a deviation from the target value (in this example, SYS is 130 mmHg and DIA is 80 mmHg) set in advance are also indicated. In the present embodiment, for the average value of the previous week and the difference from the target value, each of symbols “+” and “−” are attached to the sides of a numerical value. Instead of or in addition to this, a change in color of the characters (such as red indicating increase, and green indicating decrease) and the like may be employed. Symbols such as arrows can also be used.

In addition, for each average measured value in each period, information (Controlled or Uncontrolled) indicating whether the target value is achieved (in the present embodiment, whether the average measured value falls below the target value) is displayed. The display is not limited to characters, and a change in the color of the frame and the like may be used to indicate whether the target value is achieved. Furthermore, in the measured value transition information Tr according to the present embodiment, when it is determined that the average measured value does not have a predetermined reliability, such information is displayed in an identifiable manner. Specifically, “Insufficient data” is displayed in a column indicating whether the target value is achieved, and information indicating that the number of data is insufficient is displayed.

The information on the average measured value described above not only depicted by numerical values, but also displayed as a graph in which a range between SYS and DIA is filled in a bar shape and a relationship with a threshold line indicating a target value is visually indicated as illustrated in FIG. 5 and FIG. 6. Further, in a case where there is measurement data of a night blood pressure (BP Nocturnal) and a blood pressure measured in a medical examination room (BP Office) in addition to data for each time period of am and pm, the graph displayed includes a graph for such data. The pulse rate is also displayed on the same axis as the blood pressure. When the reliability of the average measured value does not satisfy a predetermined criterion, the graph is displayed with hatching, and thus it can be understood at a glance 10 that the data lacks reliability.

As illustrated in FIG. 5 and FIG. 7, in the medicine-related information Me of the present embodiment, information such as the type of medicine taken by the patient P in that week, the actual dosing frequency (in this example, indicated as Adherence using a dose rate), and a symptom and frequency of a complained side effect are displayed in the same time series as the measured value transition information Tr. In addition, information (Prescription) indicating prescription contents, such as a brand, a dosage form, and a dose amount of a medicine, can also be referred to.

By referring to such a screen that allows browsing the measured value transition information Tr and the medicine-related information Me, the doctor can easily and accurately recognize the effect of the prescribed medicine, and promptly determine what kind of treatment strategy should be adopted for the patient P.

### Patient-Side Terminal

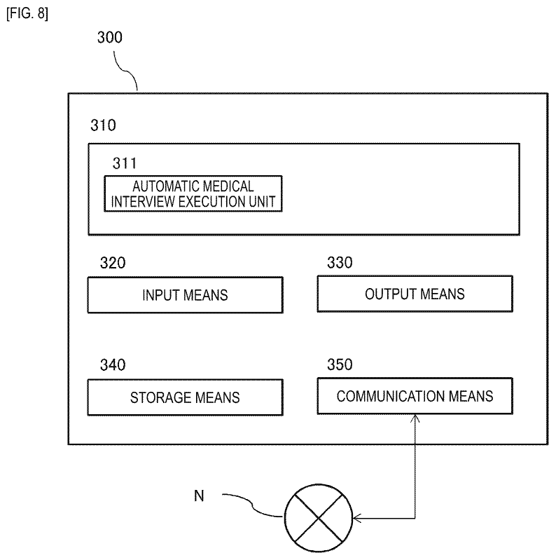

FIG. 8 is a block diagram illustrating a functional configuration of the patient-side terminal 300. The patient-side terminal 300 is, for example, a portable information processing terminal, such as a smartphone, a tablet terminal, or a wristwatch type wearable terminal, and includes a control unit 310, input means 320, output means 330, storage means 340, and communication means 350.

The control unit 310 is means for controlling the patient-side terminal 300, and is constituted of, for example, a CPU. As the input means 320, a touch panel display integrated with the output means 330 or the like can be adopted. Similarly to other terminals, the storage means 340 is constituted including a main storage unit, an auxiliary storage unit, and the like, and stores various data acquired via the communication network N, such as an operating system (OS) and various programs. The communication means 350 is constituted including, for example, a wireless communication circuit for wireless communication.

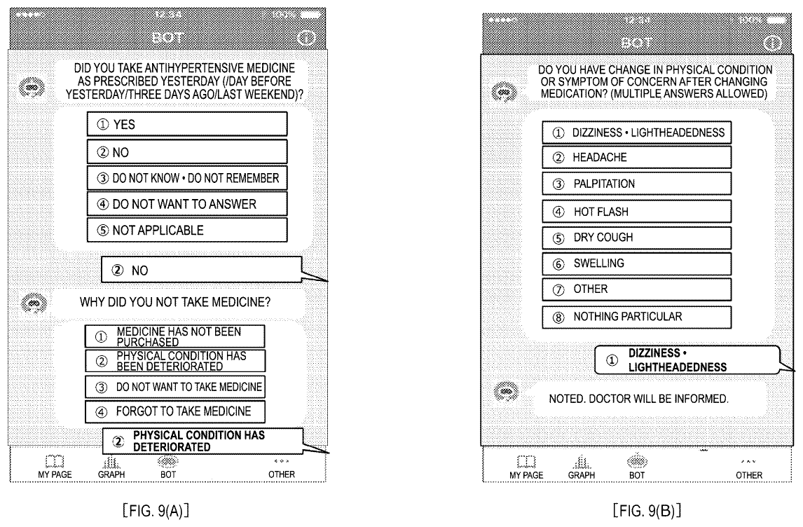

The control unit 310 includes an automatic medical interview execution unit 311 as a functional module related to dosing related information management. For example, through reception and the like of an automatic medical interview trigger signal transmitted from the server device 100, the automatic medical interview execution unit 311 executes an application for automatically performing a medical interview about the dosing frequency, the presence or absence of a side effect, and the type thereof. The automatic medical interview application can adopt a form such as a chatbot, for example, and may be stored in advance in the storage means 340 of the patient-side terminal 300 or may be provided in the form of an Application Service Provider (ASP) in the server device 100. FIG. 9(A) and FIG. 9(B) illustrate a screen example at the time of executing an automatic medical interview program. FIG. 9(A) illustrates a medical interview screen relating to medication, and FIG. 9(B) illustrates a medical interview screen relating to side effects.

The answer from the patient P to the automatic medical interview as described above is transmitted from the communication means 350 to the server device 100 via the communication network N. In addition to measurement data acquired from the blood pressure monitor 400 described below, necessary information input by the patient P is transmitted to the server device 100.

In addition, the patient-side terminal 300 receives the action evaluation information including the information related to the evaluation of the approach from the server device 100 according to the result of the approach for the treatment of the patient P, and the content is output to the output means 330.

### Blood Pressure Monitor

The blood pressure monitor 400 is used by the patient P for daily blood pressure measurement, and may have any kind of form. For example, it may be a general stationary type, a portable type in which a portable cuff is wrapped around an upper arm or the like, or may be of a wearable type to be worn on a wrist of a patient. The blood pressure is measured by the oscillometric method by an operation of the patient P (or, in the case of a wearable type, at a timing or time interval set in advance), and the blood pressure data is wirelessly transmitted to the patient-side terminal 300 by wireless communication, for example. As a communication interface used between the blood pressure monitor 400 and the patient-side terminal 300, a short-range wireless data communication standard, such as Bluetooth (trade name) or infrared communication, can be adopted.

The blood pressure monitor 400 need not have communication means. In that case, the patient P may manually input measurement data (including a blood pressure value, a pulse rate, and a measurement date and time) to the patient-side terminal 300, and the information may be transmitted to the server device 100.

Further, the patient-side terminal 300 may also have the function of the blood pressure monitor 400. For example, in a case where the patient-side terminal 300 is a wearable terminal worn on the wrist of the patient P, when a blood pressure measurement function is provided in the wearable terminal, the wearable terminal can also serve as the blood pressure monitor 400. Conversely, for example, the stationary blood pressure monitor 400 may have a function as an information processing terminal, and also serve as the patient-side terminal 300.

The measurement method of the blood pressure monitor 400 may be a method in which measurement is performed for each heartbeat, or a trigger measurement method in which blood pressure fluctuation is estimated from pulse transit time and blood pressure is measured in a spot manner using the fluctuation as a trigger.

### Flow of Information Processing in System

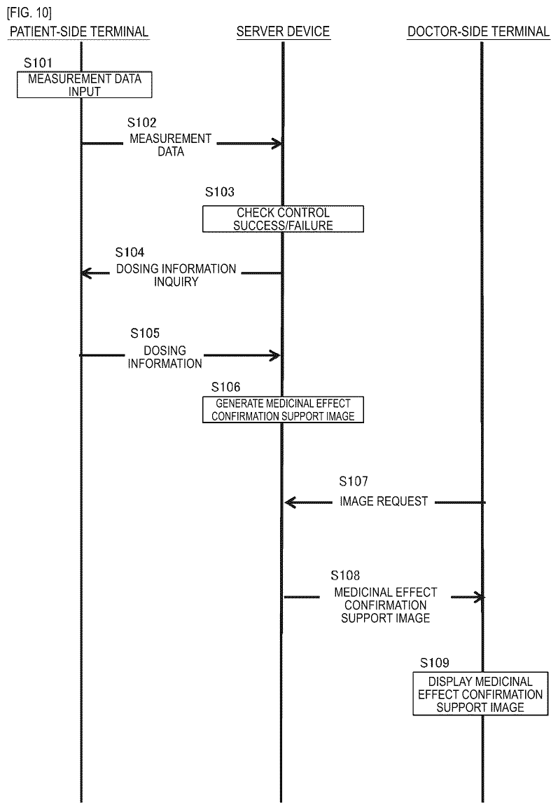

Next, a flow of information processing performed by the medical care assistance system 1 according to the present embodiment having the above-described configuration will be described. FIG. 10 is a diagram illustrating a flow of information exchange and processing performed in the medical care assistance system 1 according to the embodiment. As illustrated in FIG. 10, first, the patient P inputs to the patient-side terminal 300, measurement data obtained by measuring his/her blood pressure using the blood pressure monitor 400 (S101). The measurement data is transmitted to the server device 100 from the patient-side terminal 300 each time or collectively for a predetermined period (for example, one week) (S102).

The server device 100 stores the received measurement data in the storage means 130, and based on the information, checks whether the blood pressure of the patient P is controlled by comparing the data with predetermined target values (S103). Here, different target values are set for the systolic blood pressure and the diastolic blood pressure, and it is determined whether the average values of the systolic blood pressure and the diastolic blood pressure for each predetermined period achieve the respectively corresponding target values. When the blood pressure is not controlled (that is, either the systolic blood pressure or the diastolic blood pressure exceeds the target value), an inquiry about the dosing frequency and the like is issued to the patient-side terminal 300 (S104).

When the inquired patient P inputs the dosing information including the dosing frequency to the patient-side terminal 300, the dosing information is transmitted to the server device 100 (S105). The server device 100 generates a medicinal effect confirmation support image including information related to the transition of the blood pressure value and information related to the dosing frequency of the patient P (S106).

Then, the doctor transmits information requesting for the medicinal effect confirmation support image warning information to the server device 100 using the doctor-side terminal 200 (S107). Then, the server device 100 that has received the request provides the medicinal effect confirmation support image to the doctor-side terminal 200 (S108), and the medicinal effect confirmation support image is displayed on the output means 230 of the doctor-side terminal 200 (S109). Here, data of the medicinal effect confirmation support image may be transmitted to the doctor-side terminal 200 and stored in the storage means 240 of the doctor-side terminal 200, or may be provided in the form of an ASP and image data may be unable to be stored. The content of the medicinal effect confirmation support image is as described above.

According to the medical care assistance system 1 of the present embodiment as described above, since the medicinal effect confirmation support image indicating the transition of the average measured value in the predetermined period before and after the prescription start date (change date) can be displayed with the prescription start date (change date) used as a reference, it is possible to easily and reliably determine whether the prescription change is effective, without the effect being baffled by the daily fluctuation of the blood pressure value.

### Modified Example

In the above-described embodiment, the example of the medicinal effect confirmation support image in the case where there is no other prescription change date in the display period until four weeks elapse from the latest prescription change date (December 21) as a reference has been described. However, there may be a case where the display period includes another prescription change date. In the present modified example, an example of a medicinal effect confirmation support image in such a case will be described.

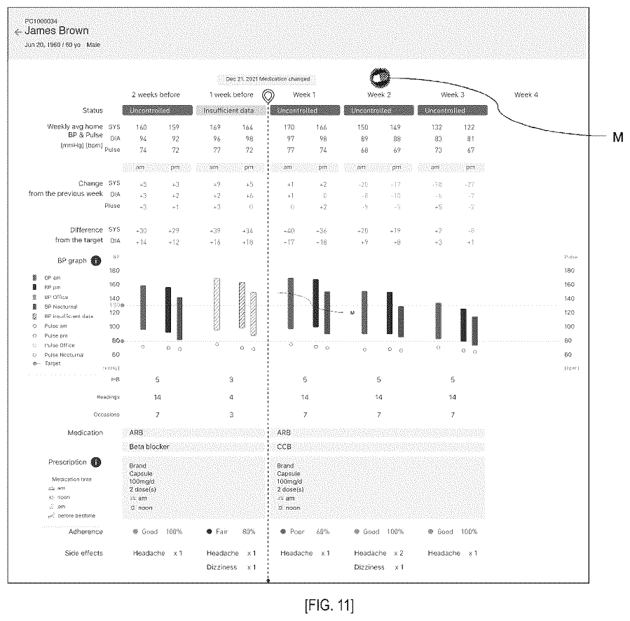

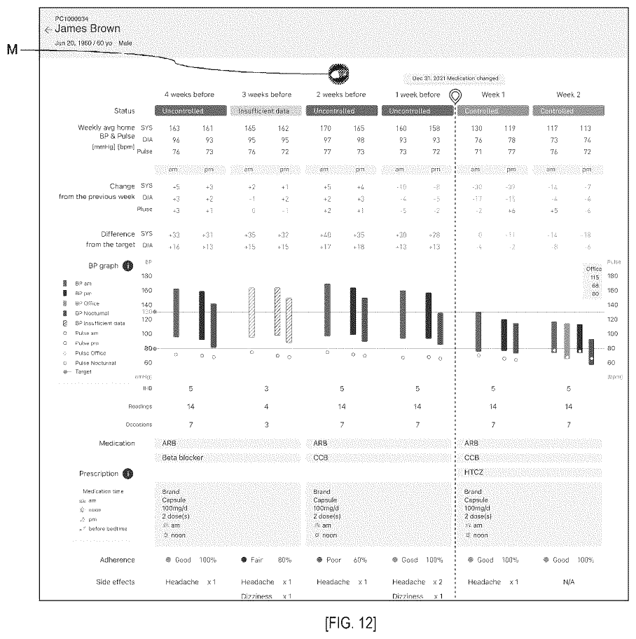

FIG. 11 and FIG. 12 are explanatory diagrams illustrating examples of the medicinal effect confirmation support image according to the present modified example. In the medicinal effect confirmation support image illustrated in FIG. 11, the measured value transition information and the medicine-related information up to January 10th of the next year are illustrated, with the prescription change made on December 21st. In the present modified example, an example in which the prescription is further changed on December 31st will be described. In such a case, as illustrated in FIG. 11, above a field indicating the week including December 31st (the second week from the change in prescription on December 21st), a prescription start date mark M indicating that the prescription with the new prescription content has started (the prescription is changed) in the week is displayed. In addition to the mark, an actual prescription start date may be displayed. Since the prescription start date mark M is displayed, the doctor can understand that the start date of the prescription with another prescription content is present within the period displayed in the medicinal effect confirmation support image.

Then, for example, by an operation such as clicking the prescription start date mark M, the date can be set as the reference date for the measured value transition information. FIG. 12 illustrates an example of a medicinal effect confirmation support image indicating measured value transition information with December 31st being the reference date. FIG. 12 illustrates an example of a redrawn (recreated) medicinal effect confirmation support image in which the predetermined period for calculating the average measured value is reset and recalculated with the reference date set to December 31st, and with the content of the medicine-related information also updated. In the image illustrated in FIG. 11, since December 31st is included in the second week, in the redrawn medicinal effect confirmation support image illustrated in FIG. 12, the new first week includes the latter half of the second week and the former half of the third week in the image example illustrated in FIG. 11, and the new second week includes the latter half of the previous third week. Then, with the predetermined period reset and the average measured value recalculated, the average measured value in the first week is displayed as “Controlled”. That is, it can be confirmed that the prescription is further changed on December 31st, and the improvement target is achieved as an effect of the prescription change.

If the medicinal effect confirmation support image in FIG. 12 further includes data that can be further displayed after the second week, the image may be displayed with the reference date slid to the left by about two weeks.

### Other Points

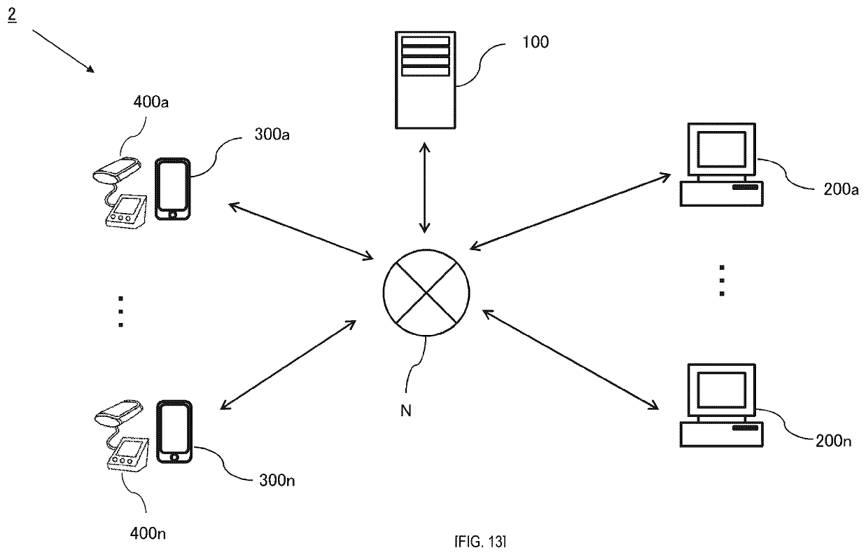

The description of each example described above is merely illustrative of the present invention, and the present invention is not limited to the specific embodiments described above. Within the scope of the technical idea of the present invention, various modifications and combinations may be made. For example, in the above-described embodiment, a configuration including one of each of the doctor-side terminal 200 and the patient-side terminal 300 has been described. However, as illustrated in FIG. 13, the present invention can be applied to a medical care assistance system 2 including a plurality of doctor-side terminals 200a to 200n and/or a plurality of patient-side terminals 300a to 300n.

Also, the means for acquiring the dosing information is not limited to the automatic medical interview program by the chatbot. For example, a sensor that detects removal of a medicine may be provided in a container of the medicine to be taken by the patient P, and the dosing information may be acquired based on information (dosing frequency, dosing amount, and the like) related to the removal of the medicine detected by the sensor. In addition, by associating with a system of a pharmacy used by the patient P, the dosing information may be acquired from information of a sold medicine, information of a sales period, or the like.

### REFERENCE NUMERALS LIST

1, 2 . . . Medical care assistance system
100 . . . Server device
110, 210, 310 . . . Control unit
120, 250, 350 . . . Communication means
130, 240, 340 . . . Storage means
200 . . . Doctor-side terminal
220, 320 . . . Input means
230, 330 . . . Output means
300 . . . Patient-side terminal
400 . . . Blood pressure monitor
P . . . Patient
N . . . Communication network
Tr . . . Measured value transition information
Me . . . Medicine-related information
M . . . Prescription start date mark

1.-15. (canceled)

16. A medical care assistance system comprising:
measured value acquisition means configured to acquire a measured value related to biological information of a patient;
medication information acquisition means configured to acquire medication information that is related to medicine prescribed to the patient, and includes information of a first prescription start date that is an earliest date of prescription of medicine with at least one prescription content;
average measured value calculation means configured to calculate an average measured value that is an average value of the measured value of the patient in each predetermined period;
reliability determination means configured to determine whether the average measured value has a predetermined reliability based on whether the number of times the biological information is measured in the predetermined period satisfies a predetermined criterion;
medicinal effect confirmation support image generation means configured to generate a medicinal effect confirmation support image including measured value transition information indicating transition of the average measured value in time series, in each of the predetermined periods before and after the first predetermined period; and
output means configured to output the medicinal effect confirmation support image, wherein
the measured value transition information in the medicinal effect confirmation support image indicates, in a distinguishable manner, the reliability of each of the average measured values.

17. The medical care assistance system according to claim 16, wherein
the measured value acquisition means further acquires information related to a time period and/or a location at which the biological information is measured,
the average measured value calculation means calculates the average measured value for each time period and/or location at which the biological information is measured, and
the measured value transition information in the medicinal effect confirmation support image indicates a change in the average measured value for each time period and/or location at which the biological information is measured.

18. The medical care assistance system according to claim 16, further comprising target achievement determination means configured to determine whether an improvement target of the biological information of the patient set in advance for each of the average measured values is achieved, wherein
the measured value transition information in the medicinal effect confirmation support image indicates information on whether the improvement target is achieved for each of the average measured values.

19. The medical care assistance system according to claim 16, further comprising change amount calculation means configured to calculate an increasing/decreasing change amount of the average measured value in one of the predetermined periods from the average measured value in an immediately preceding one of the predetermined periods, wherein
the measured value transition information in the medicinal effect confirmation support image visibly indicates the increasing/decreasing change amount for each of the average measured values, with increase and decrease being distinguishable from each other.

20. The medical care assistance system according to claim 16, wherein
the measured value acquisition means acquires, when an abnormality occurs while the biological information is being measured, information on the abnormality together with the measured value,
the average measured value calculation means calculates an average value of measured values in the predetermined period, while excluding a measured value corresponding to the occurrence of the abnormality, and
the reliability determination means determines whether the predetermined criterion is satisfied, by subtracting from the number of times of the measurement, measurement of the biological information corresponding to the occurrence of the abnormality.

21. The medical care assistance system according to claim 16, wherein
the medication information includes information related to a name of the prescribed medicine and an amount of the prescribed medicine, and
the medicinal effect confirmation support image indicates medicine-related information including the medication information in addition to the measured value transition information.

22. The medical care assistance system according to claim 21, wherein
the medication information includes, when there is a medicine prescribed to the patient with another content in addition to the medicine prescribed with the one prescription content, information related to a name and an amount of the medicine with the other content, and
when a period indicated by the measured value transition information displayed in the medicinal effect confirmation support image includes a period during which the medicine is prescribed with the other content, the medicine-related information further indicates information related to the name and the amount of the medicine in the period during which the medicine is prescribed with the other content.

23. The medical care assistance system according to claim 22, wherein
the medication information includes information of a second prescription start data that is earliest date of the prescription of the medicine with the other content, and
when a period displayed in the medicinal effect confirmation support image includes the second prescription start date, a mark indicating the second prescription start date is displayed in the medicinal effect confirmation support image.

24. The medical care assistance system according to claim 21, further comprising dosing related information acquisition means configured to acquire dosing information related to a dosing status of the patient and/or side effect information related to a side effect as a result of dosing by the patient, wherein
the medicine-related information in the medicinal effect confirmation support image indicates the dosing information and/or the side effect information.

25. The medical care assistance system according to claim 16, wherein the measured value transition information includes a graph display indicating a transition of the average measured value of the biological information.

26. The medical care assistance system according to claim 25, wherein
the measured value of the biological information includes a blood pressure value and a pulse rate, and
the graph display displays average values of the blood pressure value and the pulse rate measured in the one predetermined period on the same axis.

27. A medical care assistance device, comprising:
the measured value acquisition means;
the medication information acquisition means;
the average measured value calculation means; and
the medicinal effect confirmation support image generation means, wherein
the medical care assistance device constitutes at least a part of the medical care assistance system according to claim 16.

28. A recording medium for causing a computer to function as the medical care assistance device according to claim 27.
